(19) United States
(12) Patent Application Publication
(10) Pub. No.: US 2021/0230179 A1
(43) Pub. Date: Jul. 29, 2021

(54) THIENO[2,3-B]PYRIDINE DERIVATIVES AS EPAC INHIBITORS AND THEIR PHARMACEUTICAL USES

(71) Applicants: INSTITUT NATIONAL DE LA SANTE ET DE LA RECHERCHE MEDICALE (INSERM), Paris (FR); UNIVERSITE PAUL SABATIER TOULOUSE III, Toulouse (FR); UNIVERSITÉ PARIS-SACLAY, Orsay (FR); SAPIENZA UNIVERSITA DI ROMA, Roma (IT)

(72) Inventors: Jean-Paul BLONDEAU, Chatenay-Malabry Cedex (FR); Antonio COLUCCIA, Roma (IT); Marion LAUDETTE, Toulouse Cedex 04 (FR); Frank LEZOUALC'H, Toulouse Cedex 04 (FR)

(21) Appl. No.: 15/734,695

(22) PCT Filed: Jun. 6, 2019
(86) PCT No.: PCT/EP2019/064886

§ 371 Date: Dec. 3, 2020

(30) Foreign Application Priority Data
Jun. 6, 2018 (EP) 18305685.2

(51) Int. Cl. C07D 495/04; A61P 9/10; A61K 9/00; A61K 47/06

(52) U.S. Cl. CPC C07D 495/04; A61K 47/06; A61K 9/0019; A61P 9/10

(57) ABSTRACT

The present invention relates to thieno[2,3-b]pyridine derivatives for use in the treatment and/or the prevention of a disease selected from the group consisting of inflammation, cancer, vascular diseases, kidney diseases, cognitive disorders, pain, infections, obesity, and cardiac diseases. Indeed, the inventors found that thieno[2,3-b]pyridine derivatives of the invention are inhibitors of the Epac protein and can thus be useful for the prevention and/or treatment of diseases wherein the Epac protein is involved. Particularly, the inventors showed that thieno[2,3-b]pyridine derivatives of the invention are potent and non-competitive inhibitors of Epac and demonstrated that they also inhibit the activation of Epac downstream effectors such as Rap1 in cells.

The present invention relates to thieno[2,3-b]pyridine derivatives useful as Epac inhibitors and their pharmaceutical uses.

The second messenger cyclic AMP (cAMP) regulates diverse physiologic processes including cell growth, permeability and inflammation. The major intracellular functions of cAMP are transduced by protein kinase A (PKA) and by the more recently identified cAMP-binding proteins, Exchange Proteins directly Activated by cAMP (here-after named Epac).

There are two isoforms of the protein Epac, Epac1 and Epac2. Both proteins act as guanine-nucleotide exchange factors (GEF) for the small G proteins, Rap1 and Rap2 and function in a PKA-independent manner. The two isoforms of Epac differ in that Epac1 has a single cyclic nucleotide-binding (CNB) domain, whereas Epac2 has two CNB domains, called CNB-A and CNB-B, which are located on both sides of the DEP domain (Dishevelled, Egl-10 and Pleckstrin domain).

Epac regulates various physiological processes linked to diseases such as inflammation, cancer, vascular diseases, kidney diseases, cognitive disorders, pain, infections, obesity, and cardiac diseases.

Therefore, there is a need to provide novel Epac inhibitors. In particular, there is a need for more potent and/or more selective Epac inhibitors. There is also a need to provide Epac inhibitors that can be used in the prevention and/or the treatment of diseases wherein Epac is involved such as inflammation, cancer, vascular diseases, kidney diseases, cognitive disorders, pain, infections, obesity, and cardiac diseases.

The aim of the invention is to provide novel Epac inhibitors.

Another aim of the invention is to provide novel Epac inhibitors, which are more potent in vivo than already known in vitro Epac inhibitors such as compound CE3F4 or 8-CPT-N6-phenyl-cAMP (8-CPT-N6 or 8-(4-chlorophenylthio)-N⁶-phenyladenosine-3′,5′-cyclicmonophosphate).

Another aim of the invention is to provide novel Epac inhibitors that prevent the activation of Epac downstream effectors, in particular Epac1-induced Rap1 activation.

Another aim of the invention is to provide novel Epac inhibitors which selectivity inhibit Epac1 catalytic activity.

An aim of the present invention is to provide novel Epac inhibitors which can be useful for the prevention and/or the treatment of inflammation, cancer, vascular diseases, kidney diseases, cognitive disorders, pain, infections, obesity, and cardiac diseases.

The present invention thus relates to a compound having the following formula

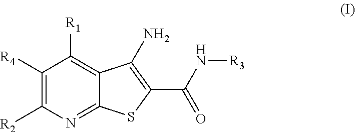

wherein:

R₁ is selected from the group consisting of:
H;
(C₂-C₂₀)alkyl;
(C₃-C₁₀)cycloalkyl;
3-10 membered heterocycloalkyl;
(C₆-C₁₀)aryl; and
5-10 membered heteroaryl;
wherein said alkyl, cycloalkyl, heterocycloalkyl, aryl and heteroaryl groups are optionally substituted;

R₂ is selected from the group consisting of:
H;
(C₁-C₂₀)alkyl;
(C₃-C₁₀)cycloalkyl;
3-10 membered heterocycloalkyl;
(C₆-C₁₀)aryl; and
5-10 membered heteroaryl;
or R₂ and R₄ together with the carbon atoms carrying them form a (C₃-C₁₀)cycloalkyl group;
wherein said alkyl, cycloalkyl, heterocycloalkyl, aryl and heteroaryl groups are optionally substituted;

R₃ is selected from the group consisting of:
H;
(C₃-C₁₀)cycloalkyl;
3-10 membered heterocycloalkyl;
(C₆-C₁₀)aryl; and
5-10 membered heteroaryl;
wherein said cycloalkyl, heterocycloalkyl, aryl and heteroaryl groups are optionally substituted; and

R₄ is selected from the group consisting of: H, —OH, —NRxRy and —C(O)ORz,

Rx, Ry and Rz being independently of each other H or a (C₁-C₁₀)alkyl;

or R₂ and R₄ together with the carbon atoms carrying them form a (C₃-C₁₀)cycloalkyl group;

or its pharmaceutically acceptable salt, hydrate or hydrated salt or its is polymorphic crystalline structure, racemate, diastereomer or enantiomer,

for use in the treatment and/or the prevention of a disease wherein the Epac protein is involved.

The inventors have surprisingly discovered that thieno[2,3-b]pyridine derivatives inhibit the Epac protein. This inhibition leads to the inhibition of the Epac-induced Rap1 activation. The inventors also surprisingly discovered that thieno[2,3-b]pyridine derivatives are non-competitive inhibitors of cAMP and of Epac agonists.

In one embodiment, the compounds of formula (I) are selective Epac inhibitors. Selective Epac inhibitors may be compounds which exhibit an inhibitory effect on the Epac protein and exhibit moderate or no inhibitory effect on other proteins. In one embodiment, the compounds of formula (I) are non-competitive inhibitors. In one embodiment, the compounds of formula (I) do not inhibit the protein kinase A (also called PKA). In another embodiment, the inhibition of Epac by the compounds of formula (I) is concentration-dependent of said compounds.

In one embodiment, the compounds of formula (I) are Epac1 inhibitors. In another embodiment, the compounds of formula (I) are Epac1 selective inhibitors. In one embodiment, Epac1 selective inhibitors are compounds which exhibit an inhibitory effect on the Epac1 isoform. More particularly, they generally exhibit an inhibitory effect on Epac1 and moderate or no inhibitory effect on Epac2 isoform. By “selective Epac1 inhibitor” it may be understood the ability of the Epac1 inhibitors to affect the particular Epac1 isoform, in preference to the other isoform Epac2. The Epac1 selective inhibitors may have the ability to discriminate between the two Epac isoforms, and so affect essentially the Epac1 isoform.

“CE3F4” refers to the compound having the following formula:

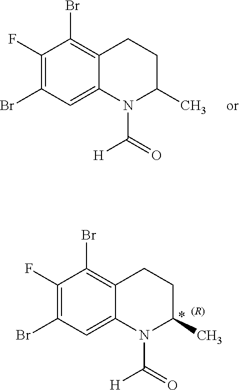

The term “(C₁-C₂₀)alkyl” or “(C₂-C₂₀)alkyl” means a saturated or unsaturated aliphatic hydrocarbon group which may be straight or branched, having 1 to 20 carbon atoms or 2 to 20 carbon atoms respectively in the chain. Preferred alkyl groups have 1 to 5 carbon atoms in the chain, preferred alkyl groups are in particular methyl or ethyl groups. “Branched” means that one or lower alkyl groups such as methyl, ethyl or propyl are attached to a linear alkyl chain. Alkyl group may be substituted.

By “(C₃-C₁₀)cycloalkyl” is meant a cyclic, saturated hydrocarbon group having 3 to 10 carbon atoms, wherein any carbon atom capable of substitution may be substituted by a substituent. In particular, cycloalkyl groups are cyclopropyl or cyclohexyl groups.

By “3-10 membered heterocycloalkyl” is meant a cyclic, saturated hydrocarbon group having 3 to 10 carbon atoms and wherein one or more carbon atom(s) are replaced by one or more heteroatom(s) such as nitrogen atom(s), oxygen atom(s) and sulfur atom(s); for example 1 or 2 nitrogen atom(s), 1 or 2 oxygen atom(s), 1 or 2 sulfur atom(s) or a combination of different heteroatoms such as 1 nitrogen atom and 1 oxygen atom. Any ring atom capable of substitution may be substituted by a substituent. Preferred 3-10 membered heterocycloalkyl are furan, thiophene, nitrogen rings such as pyrrole or pyrazole or fluorophenyl rings.

The term “(C₆-C₁₀)aryl” refers to an aromatic monocyclic, bicyclic, or tricyclic hydrocarbon ring system wherein any ring atom capable of substitution may be substituted by a substituent. Examples of aryl moieties include, but are not limited to, phenyl.

The term “5-10 membered heteroaryl” refers to an aromatic monocyclic, bicyclic, or tricyclic hydrocarbon ring system, wherein any ring atom capable of substitution may be substituted by a substituent and wherein one or more carbon atom(s) are replaced by one or more heteroatom(s) such as nitrogen atom(s), oxygen atom(s) and sulfur atom(s); for example 1 or 2 nitrogen atom(s), 1 or 2 oxygen atom(s), 1 or 2 sulfur atom(s) or a combination of different heteroatoms such as 1 nitrogen atom and 1 oxygen atom. Preferred heteroaryl groups are thienyl, pyridyl, pyrimydyl and oxazyl groups, more preferably thienyl group.

The term “halogen” refers to the atoms of the group 17 of the periodic table and includes in particular fluorine, chlorine, bromine, and iodine atoms, more preferably fluorine, chlorine and bromine atoms, for example fluorine.

By “optionally substituted”, it may be meant that the alkyl, cycloalkyl, heterocycloalkyl, aryl and heteroaryl groups of the compounds of the invention are optionally substituted by one or more substituent(s) selected from the group consisting of: —OH, halogen atom, —C(O)OH, —(C₁-C₁₀)alkyl, —(C₁-C₁₀)alkoxy, and —NR₇R₈ group, wherein R₇ and R₈ are independently of each other selected from (C₁-C₁₀)alkyl or H.

Preferably, said alkyl, cycloalkyl, heterocycloalkyl, aryl and heteroaryl groups are optionally substituted by one or more halogen atom(s), more preferably by a fluorine atom.

The terms “thieno[2,3-b]pyridine derivatives” refer to compounds derived from the following chemical structure:

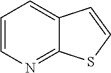

The compounds herein described may have asymmetric centers. Compounds of the present invention containing an asymmetrically substituted atom may be isolated in optically active or racemic forms. It is well-known in the art how to prepare optically active forms, such as by resolution of racemic forms or by synthesis from optically active starting materials. All chiral, diastereomeric, racemic forms and all geometric isomeric forms of a compound are intended, unless the stereochemistry or the isomeric form is specifically indicated.

The term “pharmaceutically acceptable salt” refers to salts which retain the biological effectiveness and properties of the compounds of the invention and which are not biologically or otherwise undesirable. Pharmaceutically acceptable acid addition salts may be prepared from inorganic and organic acids, while pharmaceutically acceptable base addition salts can be prepared from inorganic and organic bases. For a review of pharmaceutically acceptable salts see Berge, et al. ((1977) J. Pharm. Sd, vol. 66, 1). For example, the salts include those derived from inorganic acids such as hydrochloric, hydrobromic, sulfuric, sulfamic, phosphoric, nitric, and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, sulfanilic, fumaric, methanesulfonic, and toluenesulfonic acid and the like.

By “a disease wherein the Epac protein is involved” is meant a disease wherein the Epac protein is expressed or over-expressed, and/or mutated.

In the context of the invention, the term “treating” or “treatment”, as used herein, means reversing, alleviating, inhibiting the progress of the disorder or condition to which such term applies, or one or more symptoms of such disorder or condition.

In the context of the invention, the term “preventing” or “prevention”, as used herein, means avoiding the appearance or the progress of the disorder or condition to which such term applies, or one or more symptoms of such disorder or condition.

The present invention also relates to a compound having the following formula (I):

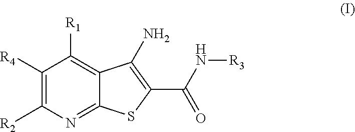

wherein:

R₁ is selected from the group consisting of:
(C₂-C₂₀)alkyl;
(C₃-C₁₀)cycloalkyl;
3-10 membered heterocycloalkyl;
(C₆-C₁₀)aryl; and
5-10 membered heteroaryl;
wherein said alkyl, cycloalkyl, heterocycloalkyl, aryl and heteroaryl groups are optionally substituted;

R₂ is selected from the group consisting of:
H;
(C₁-C₂₀)alkyl;
(C₃-C₁₀)cycloalkyl;
(C₆-C₁₀)aryl; and
5-10 membered heteroaryl;
or R₂ and R₄ together with the carbon atoms carrying them form a (C₃-C₁₀)cycloalkyl group;
wherein said alkyl, cycloalkyl, aryl and heteroaryl groups are optionally substituted;

R₃ is selected from the group consisting of:
H;
(C₃-C₁₀)cycloalkyl;
3-10 membered heterocycloalkyl;
(C₆-C₁₀)aryl; and
5-10 membered heteroaryl;
wherein said cycloalkyl, heterocycloalkyl, aryl and heteroaryl groups are optionally substituted; and

R₄ is selected from the group consisting of: H, —OH, —NRxRy and —C(O)ORz,

Rx, Ry and Rz being independently of each other H or a (C₁-C₁₀)alkyl;

or R₂ and R₄ together with the carbon atoms carrying them form a (C₃-C₁₀)cycloalkyl group;

or its pharmaceutically acceptable salt, hydrate or hydrated salt or its polymorphic crystalline structure, racemate, diastereomer or enantiomer,

for use in the treatment and/or the prevention of a cardiac disease selected from the group consisting of: cardiac hypertrophy, cardiac arrhythmias, valvulopathies, diastolic dysfunction, chronic heart failure, ischemic heart failure, myocardial ischemia, reperfusion injury, myocarditis, hypertrophic and dilated cardiomyopathies.

According to an embodiment, R₃ is selected from the group consisting of:

(C₃-C₁₀)cycloalkyl;

3-10 membered heterocycloalkyl;

(C₆-C₁₀)aryl; and

5-10 membered heteroaryl;

wherein said cycloalkyl, heterocycloalkyl, aryl and heteroaryl groups are optionally substituted.

Compounds of General Formula (I)

In one embodiment, R₃ is selected from the group consisting of:
(C₃-C₁₀)cycloalkyl;
3-10 membered heterocycloalkyl;
(C₆-C₁₀)aryl; and
5-10 membered heteroaryl;

wherein said cycloalkyl, heterocycloalkyl, aryl and heteroaryl groups are optionally substituted.

In a particular embodiment, R₃ is a (C₆-C₁₀)aryl optionally substituted by one or more substituent(s), preferably selected from the group consisting of: (C₁-C₁₀)alkyl and halogen atom. In one embodiment, R₃ is H or a (C₆-C₁₀)aryl optionally substituted by one or more substituent(s) selected from the group consisting of: (C₁-C₁₀)alkyl and halogen atom. In a particular embodiment, R₃ is H or a phenyl optionally substituted by one or more halogen atom(s). In a particular embodiment, R₃ is a phenyl optionally substituted, preferably by one or more halogen atom(s).

In another embodiment, R₁ is selected from the group consisting of:
H;
(C₆-C₁₀)aryl; and
5-10 membered heteroaryl;

wherein said aryl and heteroaryl groups are optionally substituted by one or more substituent(s) selected from the group consisting of: (C₁-C₁₀)alkyl, halogen atom and a —NR₇R₈ group; wherein R₇ and R₈ are independently of each other selected from (C₁-C₁₀)alkyl or H.

Preferably, R₁ is H or a (C₆-C₁₀)aryl optionally substituted by one or more substituent(s), for example by substituents selected from the group consisting of: (C₁-C₁₀)alkyl, halogen atom and a —NR₇R₈ group; wherein R₇ and R₈ are independently of each other selected from (C-Coo)alkyl or H. In a particular embodiment, R₁ is H or a phenyl optionally substituted by one or more halogen atom(s), for example by one fluorine atom, preferably in the para position.

In another embodiment, R₁ is selected from the group consisting of:
(C₆-C₁₀)aryl; and
5-10 membered heteroaryl;

wherein said aryl and heteroaryl groups are optionally substituted by one or more substituent(s) selected from the group consisting of: (C₁-C₁₀)alkyl, halogen atom and a —NR₇R₈ group; wherein R₇ and R₈ are independently of each other selected from (C₁-C₁₀)alkyl or H.

Preferably, R₁ is a (C₆-C₁₀)aryl optionally substituted by one or more substituent(s), for example by substituents selected from the group consisting of: (C₁-C₁₀)alkyl, halogen atom and a —NR₇R₈ group; wherein R₇ and R₈ are independently of each other selected from (C₁-C₁₀)alkyl or H. In a particular embodiment, R₁ is a phenyl optionally substituted by one or more halogen atom(s), for example by one fluorine atom, preferably in the para position.

In one embodiment, R₂ is selected from the group consisting of:
H;
(C₁-C₂₀)alkyl;
(C₆-C₁₀)aryl; and
5-10 membered heteroaryl;

or R₂ and R₄ together with the carbon atoms carrying them form a (C₃-C₁₀)cycloalkyl group;

wherein said alkyl, cycloalkyl, aryl and heteroaryl groups are optionally substituted by one or more substituent(s) selected from the group consisting of: (C₁-C₁₀)alkyl and halogen atom.

In a particular embodiment, R₂ is selected from the group consisting of: (C₁-C₁₀)alkyl, and 5-6 membered heteroaryl group or R₂ and R₄ together with the carbon atoms carrying them form a (C₃-C₆)cycloalkyl group; wherein said alkyl, cycloalkyl, and heteroaryl groups are optionally substituted, preferably by one or more substituent(s) selected from the group consisting of: (C₁-C₁₀)alkyl and halogen atom.

In a particular embodiment, R₂ is selected from the group consisting of 5-6 membered heteroaryl groups, said heteroaryl groups being optionally substituted, preferably by one or more substituent(s) selected from the group consisting of: (C₁-C₁₀)alkyl and halogen atom. In a particular embodiment, R₂ is a thienyl group.

In a particular embodiment, R₂ is selected from the group consisting of: (C₁-C₁₀)alkyl and a thienyl ring or R₂ and R₄ together with the carbon atoms carrying them form a (C₅-C₆)cycloalkyl group such as a cyclohexyl group.

In one embodiment, R₄ is selected from the group consisting of: H, —OH, —NH₂ and —C(O)OH or R₂ and R₄ together with the carbon atoms carrying them form a (C₃-C₁₀)cycloalkyl group. In one embodiment, R₄ is H or R₂ and R₄ together with the carbon atoms carrying them form a (C₅-C₆)cycloalkyl group. Preferably R₄ is H.

In a particular embodiment, R₁ is a phenyl group and/or R₂ is a thienyl group, said phenyl and thienyl groups being optionally substituted. In one embodiment, at least one of R₁ and R₂ is a (C₆-C₁₀)aryl group or a 5-10 membered heteroaryl group.

Preferably, in the above formula (I), R₁ is selected from the group consisting of: (C₂-C₂₀)alkyl; (C₃-C₁₀)cycloalkyl; 3-10 membered heterocycloalkyl; (C₆-C₁₀)aryl; and 5-10 membered heteroaryl; wherein said alkyl, cycloalkyl, heterocycloalkyl, aryl and heteroaryl groups are optionally substituted; and
R₂ is selected from the group consisting of: H; (C₁-C₂₀)alkyl; (C₃-C₁₀)cycloalkyl; (C₆-C₁₀)aryl; and 5-10 membered heteroaryl;
or R₂ and R₄ together with the carbon atoms carrying them form a (C₃-C₁₀)cycloalkyl group;
wherein said alkyl, cycloalkyl, aryl and heteroaryl groups are optionally substituted.

In one particular embodiment, the compounds of the invention are characterized by the following formula (II):

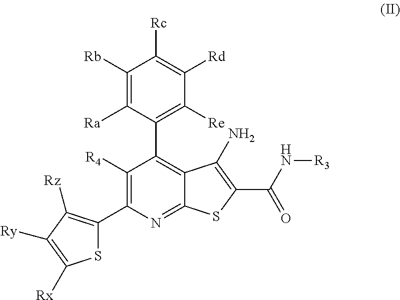

wherein Ra, Rb, Rc, Rd, Re, Rx, Ry and Rz are selected among the group is consisting of:

H, —OH, halogen atom, —C(O)OH, (C-Coo)alkyl, (C-Coo)alkoxy, and —NR₅R₆,

wherein R₅ and R₆ are independently of each other selected from (C-Coo)alkyl or H;

R₄ is selected from the group consisting of H, —OH, —NH₂ and —C(O)OH; and

R₃ is as defined above.

In a particular embodiment, Ra, Rb, Rc, Rd, Re, Rx, Ry and Rz are selected among H, halogen atom or (C-Coo)alkyl. In one embodiment, Rx, Ry and Rz are H and/or Ra, Rb, Rd and Re are H. Preferably, Rc is H or an halogen atom, for example a fluorine atom.

In one embodiment, the compound of formula (I) has one of the following formulae:

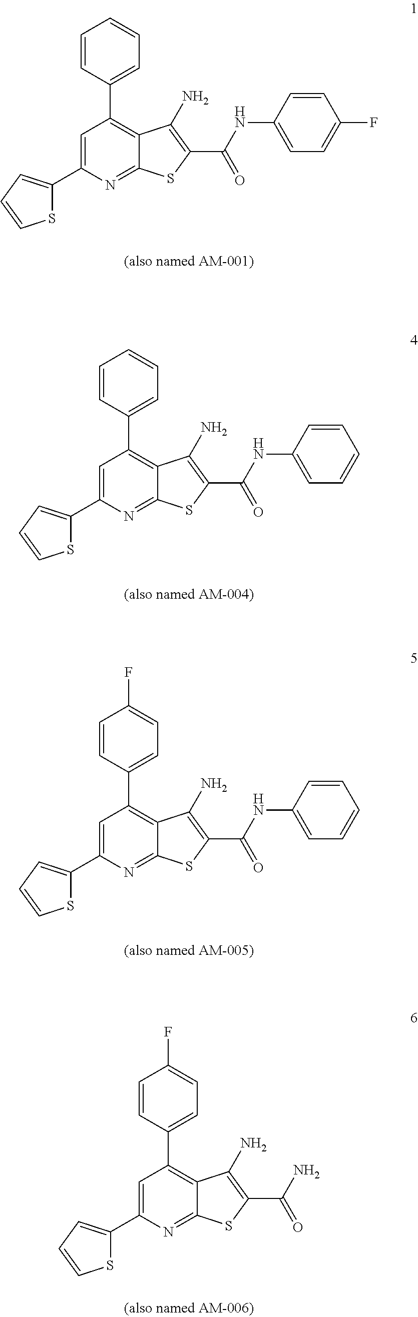 [SMILES: Nc1c(C(=O)Nc2ccc(F)cc2)sc2nc(-c3cccs3)cc(-c3ccccc3)c12 ; Nc1c(C(=O)Nc2ccccc2)sc2nc(-c3cccs3)cc(-c3ccccc3)c12 ; Nc1c(C(=O)Nc2ccccc2)sc2nc(-c3cccs3)cc(-c3ccc(F)cc3)c12 ; NC(=O)c1sc2nc(-c3cccs3)cc(-c3ccc(F)cc3)c2c1N]

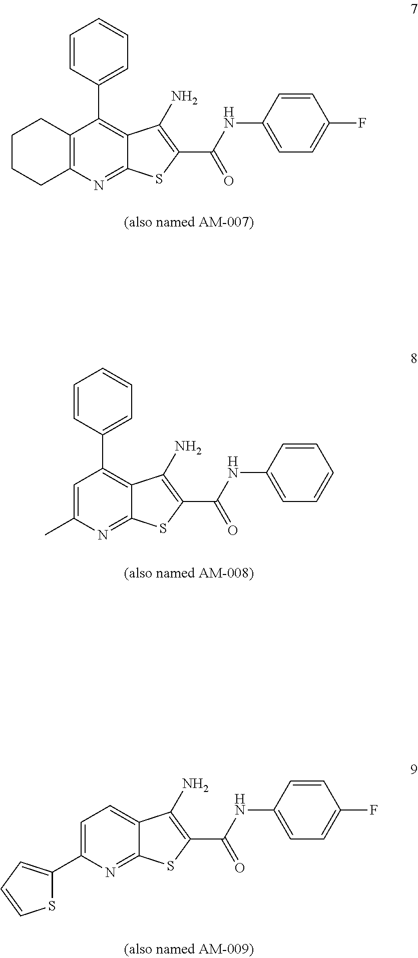 [SMILES: Nc1c(C(=O)Nc2ccc(F)cc2)sc2nc3c(c(-c4ccccc4)c12)CCCC3 ; Cc1cc(-c2ccccc2)c2c(N)c(C(=O)Nc3ccccc3)sc2n1 ; Nc1c(C(=O)Nc2ccc(F)cc2)sc2nc(-c3cccs3)ccc12]

Preferably, the compound of formula (I) has the following formula:

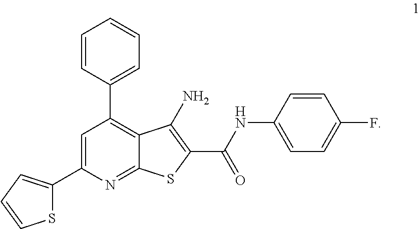 [SMILES: Nc1c(C(=O)Nc2ccc(F)cc2)sc2nc(-c3cccs3)cc(-c3ccccc3)c12]

The invention also relates to compounds of formula (I) as such.

Pharmaceutical Compositions and Uses The present invention also relates to a pharmaceutical composition, comprising a compound having formula (I) for its use as defined above, in association with at least one pharmaceutically acceptable excipient.

The present invention also relates to a drug, comprising a compound having formula (I) for its use as defined above.

While it is possible for the compounds having formula (I) to be administered alone, it is preferred to present them as pharmaceutical compositions. The pharmaceutical compositions, both for veterinary and for human use, useful according to the present invention comprise at least one compound having formula (I) as above defined, together with one or more pharmaceutically acceptable carriers and possibly other therapeutic ingredients.

In certain preferred embodiments, active ingredients necessary in combination therapy may be combined in a single pharmaceutical composition for simultaneous administration.

As used herein, the term “pharmaceutically acceptable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectables either as liquid solutions or suspensions; however, solid forms suitable for solution, or suspensions, in liquid prior to use can also be prepared. The preparation can also be emulsified. In particular, the pharmaceutical compositions may be formulated in solid dosage form, for example capsules, tablets, pills, powders, dragees or granules.

The choice of vehicle and the content of active substance in the vehicle are generally determined in accordance with the solubility and chemical properties of the active compound, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulphate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used.

The pharmaceutical compositions can be administered in a suitable formulation to humans and animals by topical or systemic administration, including oral, rectal, nasal, buccal, ocular, sublingual, transdermal, rectal, topical, vaginal, parenteral (including subcutaneous, intra-arterial, intramuscular, intravenous, intradermal, intrathecal and epidural), intracisternal and intraperitoneal. It will be appreciated that the preferred route may vary with for example the condition of the recipient.

The formulations can be prepared in unit dosage form by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

The invention relates to a compound having formula (I) as defined above, for its use for the treatment and/or the prevention of inflammation, cancer (melanoma, breast, ovarian, cervical, colon, lung, pancreatic and gastric cancers, and fibrosarcoma), vascular diseases, kidney diseases, cognitive disorders, chronic pain, bacterial and viral infections, obesity, lung (COPD, airway fibrosis) and cardiac diseases.

The invention also relates to the use of a compound having formula (I) as defined above, for the preparation of a medicament for the treatment and/or the prevention of inflammation, cancer, vascular diseases, kidney diseases, cognitive disorders, pain, infections, obesity, and cardiac diseases.

In one embodiment, cardiac diseases are selected from the group consisting of: cardiac hypertrophy, cardiac arrhythmias, valvulopathies, diastolic dysfunction, chronic heart failure, ischemic heart failure, myocardial ischemia, reperfusion injury, myocarditis, hypertrophic, dilated and diabetic cardiomyopathies. In a particular embodiment, said cardiac diseases are selected from the group consisting of hypertrophy, myocardial ischemia and reperfusion injury. In a particular embodiment, said cardiac diseases are selected from the group consisting of hypertrophy, myocardial ischemia and reperfusion injury.

By “inflammation” is meant phenomena by which the human body defends itself against aggression and which can manifest itself in various symptoms such as pain, swelling, heat or redness of the skin.

By the term “cancer” is meant solid tumors and/or disseminated hematological cancers and/or their metastasis. The terms “metastasis” or “metastatic diseases” refer to secondary tumors that are formed by cells from a primary tumor which have moved to another localization. The term “hematological cancers” refers to types of cancer that affect blood, bone marrow, and lymph nodes such as myelomas, lymphomas or leukemias. In particular, the term “cancer” refers to breast, ovarian, cervical, colon, lung, pancreatic, gastric and pancreatic cancers, melanoma, and fibrosarcoma).

By “vascular diseases” it may be meant atherogenesis, atherosclerosis and postangioplasty restenosis.

By “kidney diseases” it may be meant pathologies of the renal tubulointerstitium such as diabetic nephropathy but also Polycystic kidney disease.

Cognitive disorders are a category of mental health disorders that primarily affect learning, memory, perception, and problem solving, and include amnesia, dementia, and delirium. Among cognitive disorders, Alzheimer's disease may be cited.

By the term “pain” is meant a physical suffering or distress due to a disease, an injury or a biological process, for example an inflammatory pain. The pain can be acute or chronic, preferably a chronic inflammatory pain.

By the term “infection” is meant a viral, bacterial, parasitic or fungal infection.

Obesity is a pathology characterized by excess body fat. By the term “obese” it may be meant a patient having a body mass index (BMI) over 30 kg/m₂ (i.e. measurement obtained by dividing a person's weight in kg by the square of the person's height in meter).

Cardiac diseases more specifically point out cardiac hypertrophy, cardiac arrhythmias, valvulopathies, diastolic dysfunction, chronic heart failure, ischemic heart failure, myocardial ischemia, reperfusion injury, myocarditis, hypertrophic and dilated cardiomyopathies. It has to be noted that besides Rap1, Epac1 has been shown to activate the small GTPase H-Ras in different cell types including primary cardiomyocytes (Keiper et al., 2004; Métrich et al., 2008; Métrich et al., 2010a, 2010b; Schmidt et al., 2001).

As used herein the term “cardiac arrhythmia” is generally defined as a disturbance of the electrical activity of the heart that manifests as an abnormality in heart rate or heart rhythm. Arrhythmia is most commonly related to cardiovascular disease, and in particular, ischemic heart disease. The tem includes sinus arrhythmia, premature beat, heart block, atrial fibrillation, atrial flutter, ventricular tachycardia, ventricular fibrillation, pulsus alternans and paroxysmal tachycardia.

The invention also relates to a method of prevention and/or treatment of a disease wherein the Epac protein is involved, said method comprising the administration of a pharmaceutical acceptable amount of a compound of formula (I) as defined above to a patient in need thereof.

Preferably, the present invention relates to a method of prevention and/or treatment of inflammation, cancer, vascular diseases, kidney diseases, cognitive disorders, pain, infections, obesity, and cardiac diseases, said method comprising the administration of a pharmaceutical acceptable amount of a compound of formula (I) as defined above to a patient in need thereof.

More particularly, the invention relates to a method of prevention and/or treatment of cardiac hypertrophy, cardiac arrhythmias, valvulopathies, diastolic dysfunction, chronic heart failure, ischemic heart failure, myocardial ischemia, reperfusion injury, myocarditis, hypertrophic and dilated cardiomyopathies, said method comprising the administration of a pharmaceutical acceptable amount of a compound of formula (I) as defined above to a patient in need thereof.

### FIGURES

FIG. 1: BRET assay. AM-001, (R)-CE3F4 (20 μM) or vehicle were added to the cell extract before addition of cAMP (100 μM), and BRET ratios (mean±S.E.M from 3 wells) were measured and plotted as percent variations in BRET ratios relative to no-inhibitor control value.

FIGS. 2 and 3: BRET ratios were measured in triplicate in the presence of increasing concentrations of AM-001 or cAMP. Data were plotted as a function of the cAMP (FIG. 2) or AM-001 (FIG. 2) concentration. The curves obtained under each condition were analyzed using Graphpad Prism, yielding the maximal BRET ratio responses (expressed as percent variations in BRET ratios relative to no-inhibitor control value). Reported values are mean±S.E.M (triplicate; some error bars are masked by the symbols. ****p<0.0001 versus vehicle.

In FIG. 2, curve a corresponds to the absence of AM-001, curve b corresponds to a concentration of AM-001 of 2 μM, curve c corresponds to a concentration of AM-001 of 5 μM, curve d corresponds to a concentration of AM-001 of 10 μM, curve e corresponds to a concentration of AM-001 of 20 μM, curve f corresponds to a concentration of AM-001 of 30 μM and curve g corresponds to a concentration of AM-001 of 60 μM.

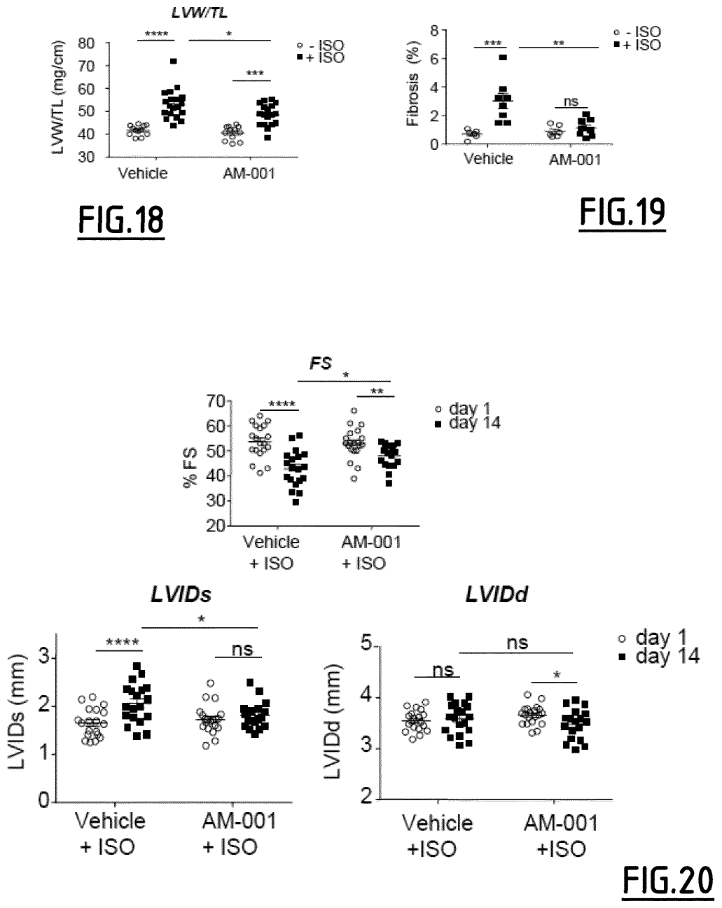

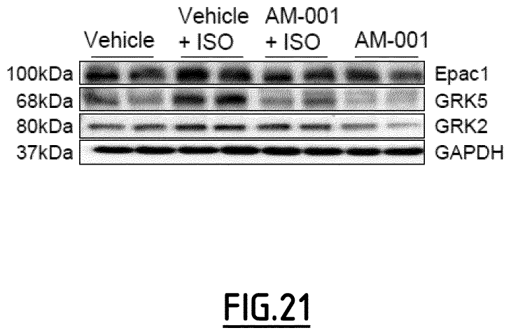

In FIG. 3, curve a corresponds to a concentration of cAMP of 1,000 μM, curve b corresponds to a concentration of cAMP of 300 μM, curve c corresponds to a concentration of cAMP of 100 μM, curve d corresponds to a concentration of cAMP of 30 μM, curve e corresponds to a concentration of cAMP of 10 μM, curve f corresponds to a concentration of cAMP of 3 μM and curve g corresponds to a concentration of cAMP of 1 μM.

FIGS. 4-8. Epac1 (FIG. 4) and Epac2 (FIG. 6) activities were measured in the absence (▪) or in the presence of 30 μM Sp-8-CPT, either alone (●) or with 20 μM (R)-CE3F4 (Δ) or with 20 μM AM-001 (⋄). Variations of relative fluorescence units (RFU) were studied as a function of time and fitted to single exponentials. Reported values are mean±S.E.M (triplicate; some error bars are masked by the symbols. (FIGS. 5 and 7) Results were expressed as the initial velocity of Epac-catalyzed GDP exchange, relative to the control values measured in the absence of inhibitor, which were set at 100%. (FIG. 8) Initial velocities of nucleotide exchange induced by Epac1 (●) and Epac2B (□) were measured in triplicate in the presence of a saturating concentration of Sp-8-CPT (50 μM) and increasing concentrations of AM-001. Results are expressed as the % of the initial velocity of GDP exchange measured in the absence of AM-001 from time course studies and are shown as mean±S.E.M (some error bars are masked by the symbols). **p<0.01, ****p<0.0001 versus control value or indicated values.

FIGS. 9-13. Amounts of Rap1-GTP (n=5 in each group, mean±S.E.M) were determined in HEK293 cells transfected with Epac1 (FIGS. 9-11), Epac2A (FIG. 12) or Epac2B (FIG. 13). Cells were preincubated or not with AM-001 (FIGS. 9, 12, and 13), AM-002 (FIG. 10) or AM-003 (FIG. 11) (20 μM, 30 min) and were then treated or not with 8-CPT-AM (10 μM for Epac1 activation and 20 μM for Epac2A activation) or S-220 (50 μM for Epac2B activation) for 10 min. ESI-05 was used as positive control of Epac2A inhibition. Representative immunoblots are shown. *p<0.01, **p<0.01, ***p<0.001, ****p<0.0001, versus indicated values, one way ANOVA, Bonferroni comparison test.

FIG. 14: Isolated adult cardiomyocytes were pretreated or not with AM-001 (20 μM, 30 min), stimulated or not with 8-CPT-AM (10 μM, 30 min), and subjected to normoxia (NX) or hypoxia-reoxygenation (HX+R) treatment. Cell viability was determined by lactate dehydrogenase (LDH) release in under NX or HX+R conditions (n=6).

FIGS. 15-16: Quantification of the area at risk (AAR) expressed as percentage of left ventricle size and infarct size expressed as percentage of AAR. Representative cross-sections stained with Evans blue and TTC of mice pretreated or not with AM-001 (FIG. 15) or R-CE3F4 (FIG. 16) are shown. Statistical significance was determined by two-way ANOVA followed by Bonferroni post-test or t-test. *p<0.01, **p<0.01, ***p<0.001, versus indicated values

FIG. 17: AST and ALT activities in the serum of mice treated under the indicated conditions (left panel) (n=7 per group). Comparison of FS in animals between day 1 and day 14 in the presence or absence of AM-001 (right panel) (n=12 per group).

FIG. 18: LVW/TL ratios of mice after 14 days of treatment with vehicle or AM-001.

FIG. 19: The bar graph shows the quantification of fibrosis (n=6-8 per group).

FIG. 20: Comparison of FS, LVIDs and LVIDd in ISO treated animals between day 1 and day 14 in the presence or absence of AM-001 (n=18-20 per group).

FIG. 21: Representative immunoblots of Epac1, GRK2, and GRK5. Statistical significance was determined by two-way ANOVA followed by Bonferroni post-test. * p<0.05, ** p<0.01, *** p<0.001, **** p<0.0001 vs indicated values. ns, not significant.

### EXAMPLES

A—Materials and Methods

Reagents and Antibodies

AM-001 and structural analogues (AM-002; AM-003; AM-004; AM-005; AM-006; AM-007; AM-008; AM-009; AM-010) were purchased from Ambinter with the following references: Amb1781597 (AM-001), Amb21993172 (AM-002), Amb2654874 (AM-003), Amb2675718 (AM-004), Amb1858954 (AM-005), Amb695840 (AM-006), Amb5842163 (AM-007), Amb3443920 (AM-008), Amb2823810 (AM-009) and Amb6326948 (AM-010).

AM-001, AM-004, AM-005, AM-006, AM-007, AM-008, and AM-009 are as described above and are compounds of formula (I) according to the present invention, whereas AM-002, AM-003, and AM-010 are not compounds according to the invention:

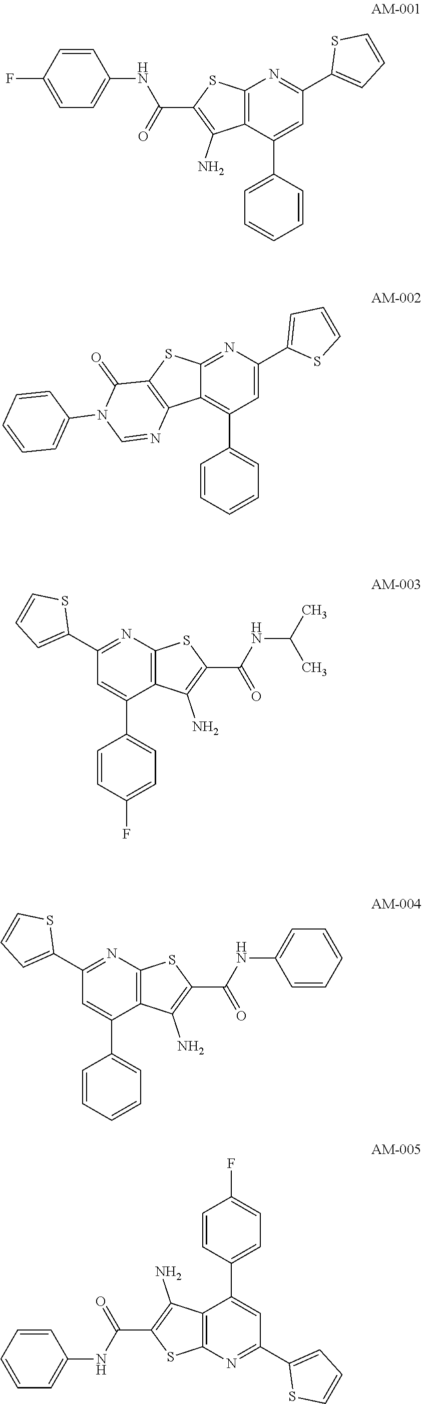 [SMILES: Nc1c(C(=O)Nc2ccc(F)cc2)sc2nc(-c3cccs3)cc(-c3ccccc3)c12 ; O=c1c2sc3nc(-c4cccs4)cc(-c4ccccc4)c3c2ncn1-c1ccccc1 ; CC(C)NC(=O)c1sc2nc(-c3cccs3)cc(-c3ccc(F)cc3)c2c1N ; Nc1c(C(=O)Nc2ccccc2)sc2nc(-c3cccs3)cc(-c3ccccc3)c12 ; Nc1c(C(=O)Nc2ccccc2)sc2nc(-c3cccs3)cc(-c3ccc(F)cc3)c12]

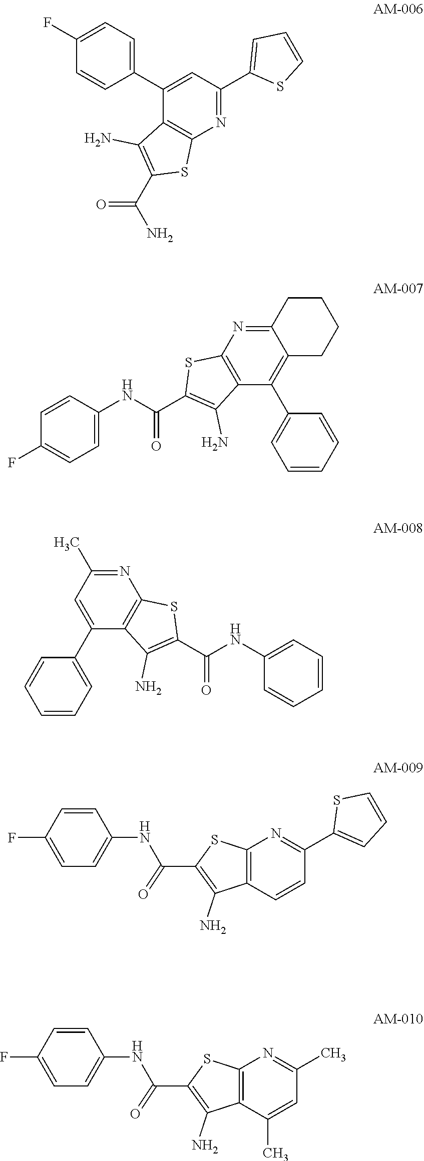 [SMILES: NC(=O)c1sc2nc(-c3cccs3)cc(-c3ccc(F)cc3)c2c1N ; Nc1c(C(=O)Nc2ccc(F)cc2)sc2nc3c(c(-c4ccccc4)c12)CCCC3 ; Cc1cc(-c2ccccc2)c2c(N)c(C(=O)Nc3ccccc3)sc2n1 ; Nc1c(C(=O)Nc2ccc(F)cc2)sc2nc(-c3cccs3)ccc12 ; Cc1cc(C)c2c(N)c(C(=O)Nc3ccc(F)cc3)sc2n1]

The Epac1 inhibitor CE3F4 was synthesized according to the methods published previously (Courilleau D, Bisserier M, Jullian J C, Lucas A, Bouyssou P, Fischmeister R, Blondeau J P, Lezoualc'h F (2012) Identification of a tetrahydroquinoline analog as a pharmacological inhibitor of the cAMP-binding protein Epac. J Biol Chem 287:44192-44202).

cAMP, 8-CPT-N6-phenyl-cAMP (8-CPT-N6), 8-(4-chlorophenylthio)-2′-O-methyladenosine-3′,5′-cyclic monophosphate, acetoxymethyl ester (8-CPT-AM) and Sp-8-CPT (Sp-8-pCPT-2′-O-Me-cAMPS (Sp-8-CPT: 8-(4-Chlorophenylthio)-2′-O-methyladenosine-3′,5′-cyclic monophosphorothioate, Sp-isomer) were purchased from BioLog (Bremen, Germany).

Guanosine 5′-diphosphate, BODIPY FL 2′-(or-3′)-O—(N-(2-aminoethyl)urethane), bis(triethylammonium) salt (bGDP) was from Invitrogen.

Antibodies and their suppliers were: anti-GAPDH, anti-Rap1, anti-Epac1, anti-Epac2 and anti-GRK2 ( 1/1000) from Cell Signaling; anti-Tubulin ( 1/5000) from Sigma-Aldrich; anti-GRK5 ( 1/1000) from Santa Cruz), Horseradish peroxidase-conjugated secondary antibodies (Santa Cruz).

Cell Culture

All procedures were performed in accordance with the Guide for the care and use of laboratory animals and the veterinary committee has been informed of the cardiac myocyte isolation protocol used

Neonatal rat ventricular myocytes (NRVM) isolation: Neonatal rats of 1-2 days old were euthanized by decapitation. The heart was excised and the atria were removed. Primary culture of NRVMs was subsequently performed as previously described (Morel E, Marcantoni A, Gastineau M, Birkedal R, Rochais F, Gamier A, Lompre A M, Vandecasteele G, Lezoualc'h F (2005) cAMP-binding protein Epac induces cardiomyocyte hypertrophy. Circ Res 97:1296-1304).

Adult murine cardiomyocytes isolation: After intraperitoneal injection of pentobarbital (300 mg/kg) and heparin (150 units), the hearts from WT and Epac1^{-/-} were rapidly isolated and placed in ice-cold Tyrode calcium free buffer (130 mmol/L NaCl, 5.4 mmol/L KCl, 1.4 mmol/L MgCl₂, 0.4 mmol/L NaH₂PO₄, 4.2 mmol/L HEPES, 10 mmol/L glucose, 20 mmol/L taurine and 10 mmol/L creatine monohydrate, pH 7.2). Primary culture of adult murine cardiomyocytes was subsequently performed as previously described (Fazal L, Laudette M, Paula-Gomes S, Pons S, Conte C, Tortosa F, Sicard P, Sainte-Marie Y, Bisserier M, Lairez O, Lucas A, Roy J, Ghaleh B, Fauconnier J, Mialet-Perez J, Lezoualc'h F (2017) Multifunctional Mitochondrial Epac1 Controls Myocardial Cell Death. Circ Res 120(4):645-657).

Human Embryonic Kidney cells line (HEK293): HEK293 cells were maintained is in minimal essential medium with FBS (10%) and penicillin-streptomycin (1%). All media, sera, and antibiotics used in cell culture were purchased from Invitrogen.

Plasmid Constructs and Transfection

The Epac1-BRET sensor CAMYEL was constructed from the pQE30-CAMYEL prokaryotic expression vector (a gift from Dr L. 1. Jiang) as previously described (Courilleau D, Bisserier M, Jullian J C, Lucas A, Bouyssou P, Fischmeister R, Blondeau J P, Lezoualc'h F (2012) Identification of a tetrahydroquinoline analog as a pharmacological inhibitor of the cAMP-binding protein Epac. J Biol Chem 287:44192-44202). The human Epac1/Epac2 expression vector was a gift from Dr J. L. Bos. Cells were transfected using Lipofectamine 2000 (Invitrogen) according to the manufacturer's instructions. HEK293 cells were maintained in MEM with FBS (Foetal Bovine Serum; 10%) and penicillin-streptomycin (1%). All media, sera and antibiotics used in cell culture were purchased from Invitrogen (Cergy Pontoise, France). Transient transfection experiments were performed with Lipofectamine 2000 (Invitrogen Life Technologies) in the presence of various amounts of plasmid constructs according to the manufacturer's instructions.

Animals

All animal procedures were performed in accordance with Institutional Guidelines on Animal Experimentation and with a French Ministry of Agriculture license. Moreover, this investigation conformed to the Guide for the Care and Use of Laboratory Animals published by the Directive 2010/63/EU of the European Parliament. Mice were housed in a pathogen-free facility, and all animal experiments were approved by the Animal Care and Use Committees of the University of Toulouse.

Myocardial I/R In Vivo, Assessment of Area at Risk and Infarct Size

Mice were anesthetized with intraperitoneal ketamine (60 mg/kg) and xylazine (6 mg/kg), intubated and ventilated mechanically. Anesthesia was maintained throughout the chirurgical procedure with 1.5% isoflurane. Body temperature was maintained at 37° C. A left thoracotomy was performed in order to enable coronary artery occlusion with a 7-0 Prolene thread placed around the left coronary artery for 45 min, followed by 24 h of reperfusion. Mice were treated with a bolus of AM-001 (10 mg/kg) or sterile saline solution 5 min before the reperfusion by intravenous injection in the coronary artery. Myocardial ischemia was confirmed by the presence of regional cyanosis. Reperfusion was confirmed by visualization of a hyperemic response. At this point, the chest was closed in layers. 24 h after reperfusion, mice were anesthetized again, the coronary artery was reoccluded at the previous site, and the heart was excised after Evans blue perfusion. The area at risk and the infarct area were respectively determined by Evans blue and 2,3,5-triphenyltetrazolium chloride (TTC) staining. The area at risk was identified as the non-blue region and expressed as a percentage of the left ventricle weight. The infarcted area was identified as the TTC negative zone and expressed as a percentage of area at risk.

Chronic ISO Infusion

Osmotic mini-pumps (Alzet) were implanted subcutaneously in mice anaesthetized with isoflurane (1%). Pumps were filled with ISO or sterile saline and were set to deliver ISO at 60 mg/kg per day for 14 days to induce cardiac hypertrophy. AM-001 treatment (10 mg/kg/d diluted in 10% DMSO/20% Kolliphor/70% sterile saline) is given from the third day of ISO infusion. Mice were subsequently euthanized with barbiturate overdose (pentobarbital, 150 mg/kg, i.p.) and mini-pumps were weighed in order to verify complete diffusion.

Echocardiograhy

Echocardiography was carried out on lightly anaesthetized (1% isoflurane in air) mice placed on a heating pad. The left ventricle dimensions were obtained during TimeMouvement mode acquisition from the parasternal short-axis view at the midventricular level of the papillary muscles using a Vevo2100 ivid7 echograph and a 4014 MHz transducer (M550i13L, VisualsonicsGE Healthcare). Images were transferred and analysed off line with Vevolab software EchoPAC (GE Healthcare).

The operator was blind to the treatment group genotype of the mice.

Determination of fibrosis Hearts were transversely sectioned at 10 μm thickness, fixed with 4% paraformaladehyde and stained with Sirius Red. Slides were scanned with NanoZoomer (Hamamatsu v1.2) and fibrosis was measured as positively stained area with Sirius Red and expressed as percent of total area, using ImageJ software (RSB).

Haematoxylin & Eosin Staining

Hearts were collected, fixed in 4% paraformaldehyde, dehydrated, and embedded in OCT tissue embedding compound (Tissue Tek, EMS). Longitudinal sections were performed at 10 μm in thickness and stained with hematoxylin and eosin for histological examination.

Hepatotoxicity Analysis

Aspartate aminotransferase (AST) and alanine aminotransferase (ALT) were measured in serum to determine the cytotoxicity, using specific kits purchased from ScienCell Research Laboratories following the manufacturer's instructions.

Data Analysis

Data are expressed as mean±S.E.M. Differences in quantitative variables were examined by one-way or two-way analysis of variance followed by post hoc test with Bonferroni correction using Graphpad Prism. Statistical significance was set to p<0.05. EC₅₀ and IC₅₀ values were computed according to a three-parameters dose-response model and compared on the basis of the extra sum-of square F test, using Graphpad Prism.

B—Results

1. Inhibition of an Epac1-Based BRET Sensor for cAMP

CAMYEL has been used, which is the established in vitro assay system based on the Epac1-BRET (bioluminescence resonance energy transfer) sensor (Jiang L I, Collins J, Davis R, Lin K M, DeCamp D, Roach T, Hsueh R, Rebres R A, Ross E M, Taussig R, Fraser I, Sternweis P C (2007) Use of a cAMP BRET sensor to characterize a novel regulation of cAMP by the sphingosine 1-phosphate/G13 pathway. J Biol Chem 282(14):10576-10584), to screen a diverse in-house chemical collection. The CAMYEL probe is composed of Epac1 inserted between Renilla luciferase and citrine, and takes advantage of the conformational changes in Epac1 that are induced upon binding of cAMP as a means to assess Epac1 activation. Upon binding of cAMP, Epac1 undergoes conformational changes that result in a is decrease of energy transfer due to luciferase moving away from citrine.

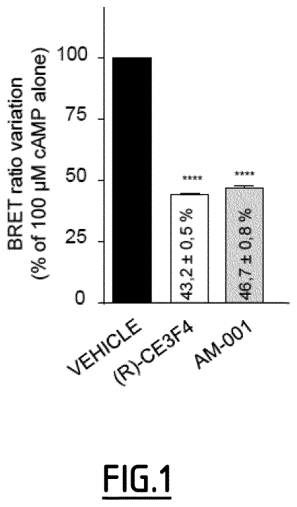

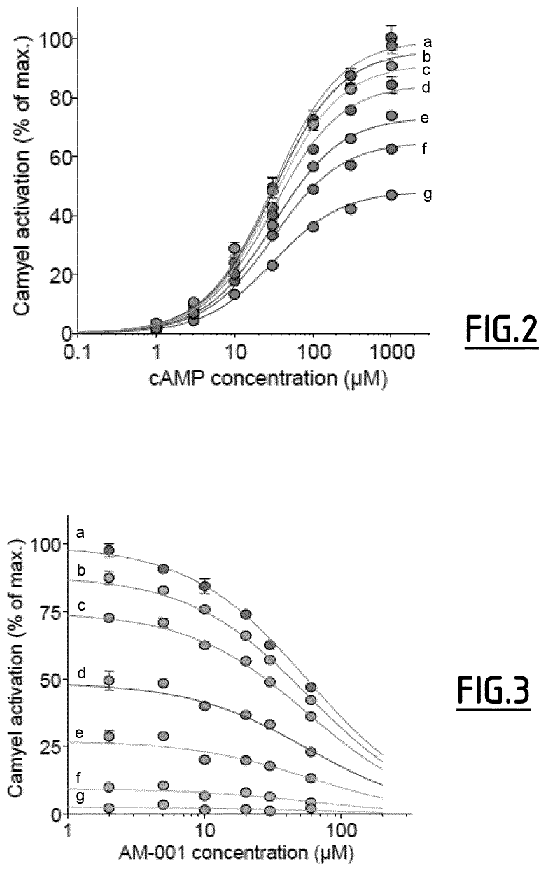

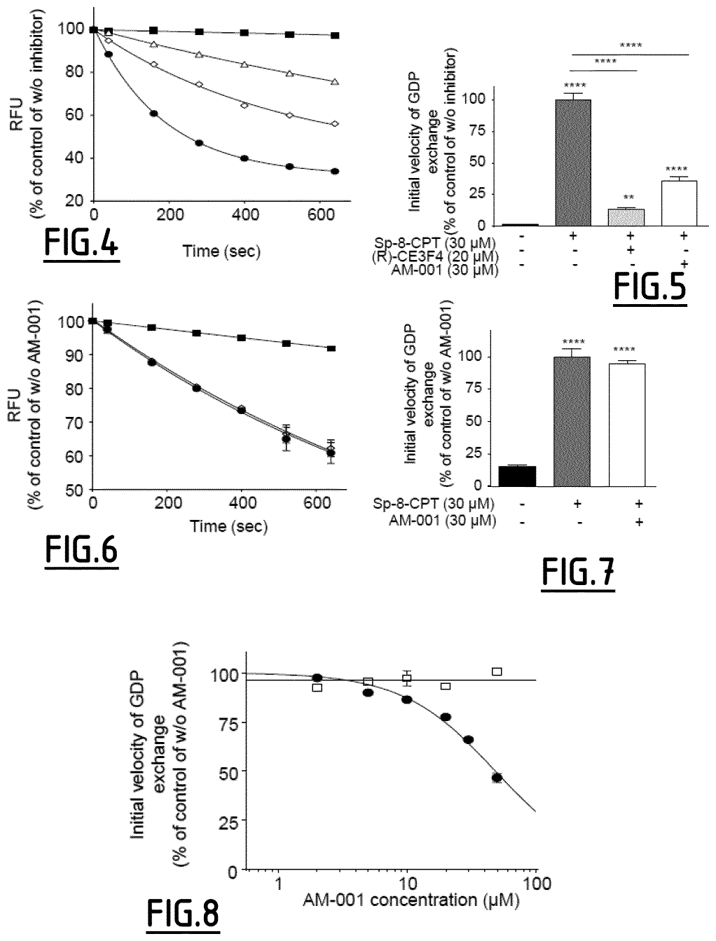

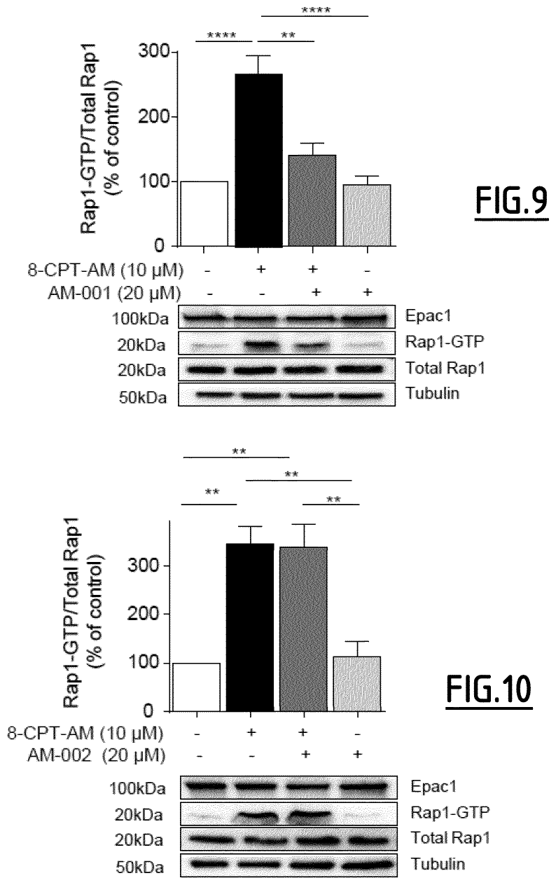

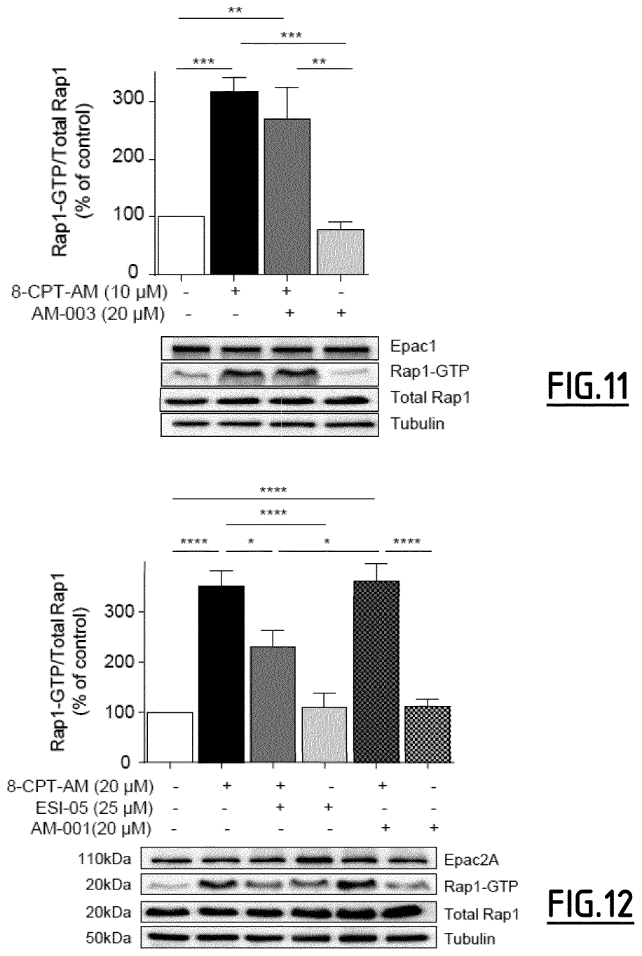

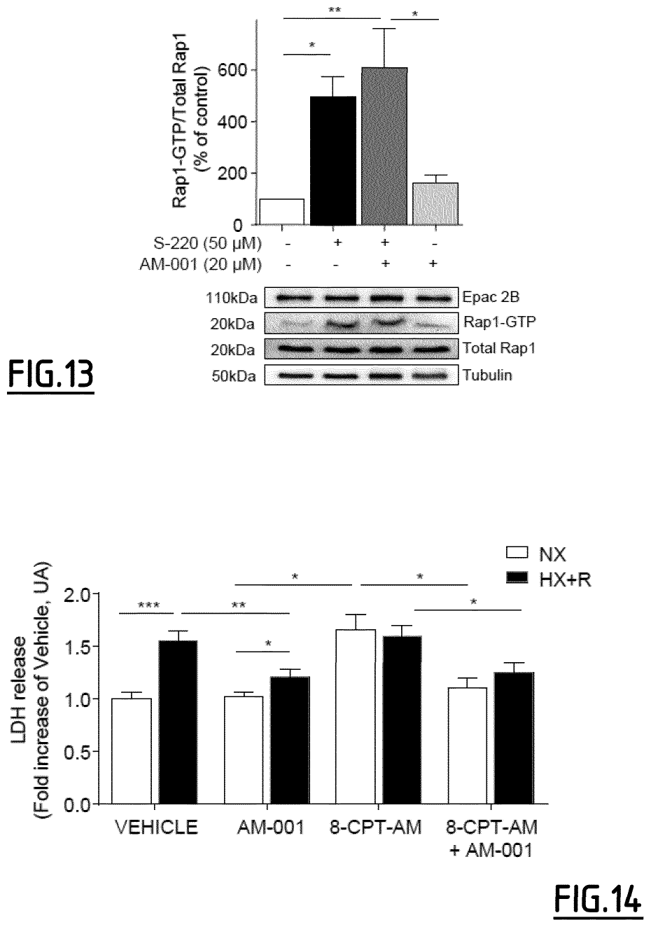

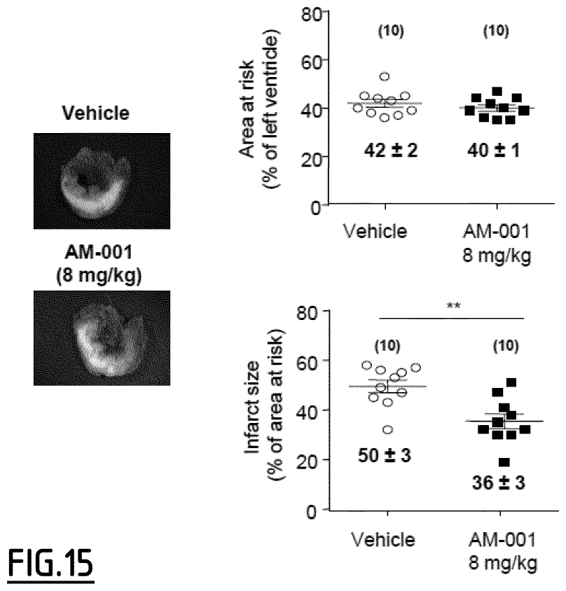

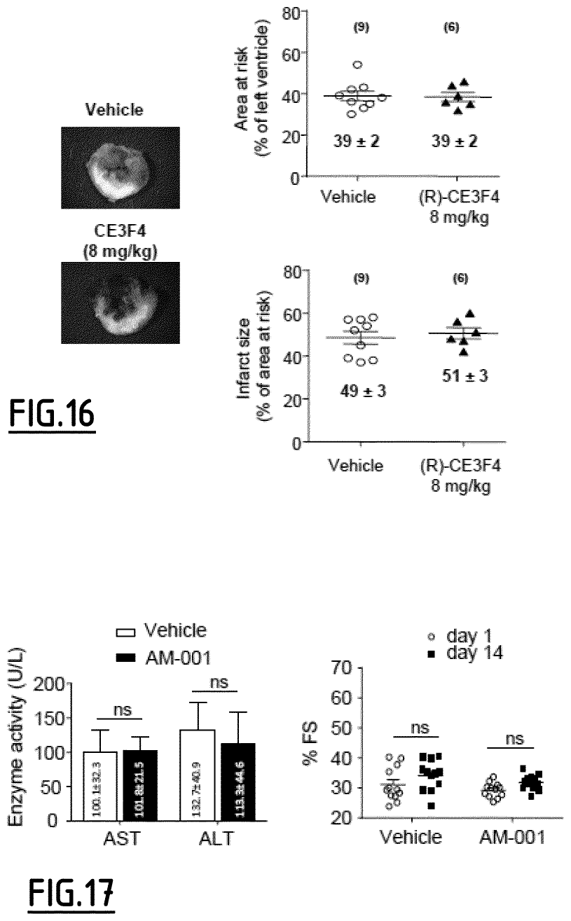

Compound 1, also named AM-001, is the 3-amino-N-(4-fluorophenyl)-4-phenyl-6-(thiophen-2-yl)thieno[2,3-b]pyridine-2-carboxamide), and it has been identified as a novel inhibitor of the cAMP-induced CAMYEL conformational change. AM-001 (20 μM) inhibited the BRET variations induced by 100 μM cAMP (≈50% decrease) in the same order of magnitude of (R)-CE3F4 (20 μM), an Epac1 uncompetitive inhibitor that was used as a standard in previous experiments (FIG. 1). AM-001 decreased BRET maximal response in a concentration dependent-manner, but not the effective concentration of cAMP that gave half-maximal response (EC₅₀=30.7±1.0 μM) (FIG. 2). Furthermore, increasing concentrations of cAMP did not modify the half-maximal inhibitory concentration (IC₅₀) of AM-001 (53.7±1.0 μM), suggesting that AM-001 behaves as a non-competitive inhibitor of Epac1 (FIG. 3). 2. AM-001 inhibits Epac1 but not Epac2 catalytic activity in vitro Next, the effects of AM-001 were investigated on Epac1 and Epac2 catalytic activities in vitro using the Epac GEF activity assay (Courilleau D, Bisserier M, Jullian J C, Lucas A, Bouyssou P, Fischmeister R, Blondeau J P, Lezoualc'h F (2012) Identification of a tetrahydroquinoline analog as a pharmacological inhibitor of the cAMP-binding protein Epac. J Biol Chem 287:44192-44202). The method is based on the Epac-stimulated dissociation of fluorescent b-GDP from recombinant Rap1 in the presence of an excess of non-fluorescent GDP. The initial velocity of the decay in fluorescence reflects exchange activity. First, we analysed if AM-001 physico-chemical properties were compatible with the Epac GEF assay. We found a linear correlation between AM-001 absorbance and concentration indicating that AM-001 was soluble up to 60 μM in the exchange reaction buffer. In addition, UV spectra of AM-001 at different concentrations showed that this compound did not absorb in the fluorescence excitation/emission window of bGDP. Importantly, the emission fluorescence intensity of bGDP was not affected when AM-001 was used at concentrations up to 60 μM. Taken together, these data showed that AM-001 physico-chemical properties were compatible with the Epac GEF assay.

Measurement of initial velocities of exchange at various concentrations of Sp-8-CPT (8-(4-chlorophenylthio)-2′-O-methyladenosine-3′,5′-cyclic mono-phosphorothioate, Sp-isomer), a strong PDE-resistant, membrane-permeant, Epac agonist gave EC₅₀ values of 7.5±1.1 μM and 5.6±1.1 μM for Epac1 and Epac2, respectively. In addition, cAMP EC₅₀ values were 13.1±1.1 μM and 2.8±1.1 μM for Epac1 and Epac2, respectively. Consequently, the apparent affinity of Epac1 was 1.7 times higher for Sp-8-CPT than for cAMP and the Vmax of Sp-8-CPT-activated Epac1 was 2.5-fold higher than the Vmax of cAMP activated Epac1. In contrast, the apparent affinity of Epac2 was 2 times higher for cAMP than for Sp-8-CPT and the Vmax of cAMP-activated Epac2 was 1.3-fold larger than the Vmax of Sp-8-CPT-activated Epac2. Therefore, a saturating concentration of Sp-8-CPT (30 μM) was chosen for the subsequent inhibitory experiments. Both Epac isoforms induced a rapid decrease in fluorescence in the presence of 30 μM Sp-8-CPT (FIGS. 4 and 6). As previously reported, the exchange activity of Epac1 induced by the agonist (30 μM Sp-8-CPT) was reduced by (R)-CE3F4 (20 μM) (FIGS. 4 and 5). AM-001 prevented Epac1 GEF activity (FIGS. 4, 5 and 8) whereas it had no inhibitory effect on Epac2 GEF activity (FIGS. 6, 7 and 8). In the presence of a saturating concentration of Sp-8-CPT, the AM-001 IC50 value was 48.5±1.0 μM for Epac1 but could not be quantified for Epac2 (IC₅₀>>1000 μM) indicating that AM-001 was ineffective in suppressing Epac2 GEF activity (FIG. 7). Similar results (AM-001 IC50=47.8±1.1 μM for Epac1 vs IC50>>1000 μM for Epac2) were obtained using concentrations of Sp-8-CPT corresponding to the EC50 for each Epac isoforms. The fact that the IC50 of inhibition of Epac1 by AM-001 was independent of the agonist concentration confirmed the non-competitive behaviour of AM-001. In addition, AM-001 decreased the maximal Epac1 GEF activity in a concentration dependent-manner, but not the EC50 of Sp-8-CPT. These results confirmed that AM-001 acted as an Epac1 non-competitive inhibitor, as already suggested by the BRET sensor experiments (FIGS. 2 and 3).

The inventors also found that AM-001 (30 μM) did not influence type I and type II PKA holoenzyme activation in the presence and absence of 10 μM cAMP, further strengthening the specificity of this compound for Epac1. Finally, using a thermal shift assay that allows monitoring of protein denaturation, it was observed that addition of AM-001 did not modify the melting temperature Tm (defined as the midpoints of thermo-unfolding) of Epac1, in the presence of increasing concentrations of AM-001, whether the agonist Sp-8-CPT was present or not, thereby excluding any non-specific protein denaturing action of AM-001.

3. Specificity of AM-001 Derivatives on Epac1 Activation

Several commercially available analogues or derivatives of AM-001 were then studied for their inhibitory action on Epac1 activation, using the CAMYEL assay system.

The results for the compounds of formula (I) are shown in the here-below is Table 1.

| | BRET ratio variation |
|---|---|
| Name | (% of 100 μM cAMP alone) |
| AM-001 | 46.7 +/− 0.8% |
| AM-002 (comp.) | 93.6 +/− 0.9% |
| AM-003 (comp.) | 100.4 +/− 2.1% |
| AM-004 | 47.0 +/− 0.7% |
| AM-005 | 46.9 +/− 1.3% |
| AM-006 | 74.0 +/− 2.8% |
| AM-007 | 80.5 +/− 3.9% |
| AM-008 | 82.2 +/− 5.5% |
| AM-009 | 82.4 +/− 1.8% |
| AM-010 (comp.) | 101.3 +/− 2.8% |

For Table 1, 20 μM of the tested compound was added to the HEK cell extract before injection of cAMP (100 μM) and BRET ratios (mean +/− S.E.M. from 3 wells) were measured and plotted as percent variations in BRET ratios relative to no-inhibitor control value.

Swapping the fluorine atom from the N-phenyl group to the 4-phenyl group (AM-005), as well as the absence of any fluorine atom on these phenyl groups (AM-004), had no significant effect on the inhibitory potency of the compounds. The inhibitory activity of compounds lacking the 4-phenyl group (AM-009) or the N-phenyl group (AM-006) was decreased but not abolished. However, substitution of the N-phenyl group by a N-isopropyl group (AM-003) abolished the inhibitory effect. The low activity of AM-010 points to the importance of the presence of the 6-thienyl group for inhibitory activity. Finally no significant inhibitory activity was observed with AM-002, a compound in which the 2-carboxamide bond cannot freely rotate, and in which the primary amino group of AM-001 is involved in an additional thieno[3,2-d]pyrimidine scaffold. This preliminary structure-activity study showed that the phenyl groups and the 6-thienyl group were essential for AM-001 to exert its inhibitory activity. Therefore, our data indicated that there was chemical specificity within this family of compounds in their ability to reduce the cAMP-induced Epac1 conformational change.

4. AM-001 Inhibits Epac1 Signaling in Cultured Cells

AM-001 displayed no apparent cytotoxicity in various types of cultured cells including neonatal rat ventricular myocytes. To confirm that AM-001 was an efficient Epac1 isoform-specific antagonist in cultured cells, we further tested its ability to block the activation of the Epac downstream effector, Rap1 in HEK293 cells (FIGS. 9-13). This cell line is commonly used for the evaluation of Epac pharmacological modulators on the cellular signaling of ectopically expressed Epac1 and Epac2 isoforms. The highly membrane-permeant and metabolically activatable Epac agonist 8-CPT-AM (8-(4-chlorophenylthio)-2′-O-methyladenosine-3′,5′-cyclic monophosphate, acetoxymethyl ester) induced a robust activation of Rap1 in cells overexpressing Epac1. Consistent with the data obtained in vitro with Epac1 BRET sensor and Epac1 exchange reaction, AM-001, but not the inactive analogues AM-002 and AM-003, prevented Epac1-induced Rap1 activation (FIGS. 9-11). Next, the inventors investigated the effect of 8-CPT-AM on the two Epac2 splice variants, Epac2A and Epac2B (FIGS. 12-13). In contrast to ESI-05, a previously characterized specific Epac2 inhibitor (Tsalkova T, Mei F C, Cheng X (2012) A Fluorescence-Based High-Throughput Assay for the Discovery of Exchange Protein Directly Activated by Cyclic AMP (EPAC) Antagonists. PLoS One 7(1): e30441), AM-001 failed to inhibit Epac2A-induced Rap1 activation (FIG. 12). Moreover, AM-001 was ineffective to impede the increase in the amount of Rap1-GTP induced by S-220 in Epac2B-transfected cells. Altogether, these data showed that AM-001 is efficient and specific in preventing Epac1-induced Rap1 activation in cultured cells.

5. AM-001 Protects Against Ischemia-Reperfusion (I/R) Injury

The inventors next examined whether AM-001 could affect the activity of Epac1 in physiologically relevant systems. Having previously shown that the genetic ablation of Epac1 prevented cardiomyocyte death during ischemic conditions (Fazal L, Laudette M, Paula-Gomes S, Pons S, Conte C, Tortosa F, Sicard P, Sainte-Marie Y, Bisserier M, Lairez O, Lucas A, Roy J, Ghaleh B, Fauconnier J, Mialet-Perez J, Lezoualc'h F (2017) Multifunctional Mitochondrial Epac1 Controls Myocardial Cell Death. Circ Res 120(4):645-657), we tested the potential protective effects of AM-001 against hypoxia-reoxygenation (HX+R) injury. Compared to control cells, AM-001 significantly increased cardiomyocyte survival as assayed by LDH release in HX+R condition (FIG. 14). Furthermore, AM-001 inhibited the effect of the membrane-permeant Epac1-specific agonist, 8-pCPT-2′-O-MecAMP-AM (8-CPT-AM) on cardiomyocyte death and hypertrophy (FIG. 14), highlighting the ability of AM-001 to counteract Epac1 detrimental effect.

Then, the therapeutic efficacy of an acute administration of AM-001 was investigated in a mouse model of acute myocardial I/R injury. A single bolus of AM-001 (8 mg/kg) or vehicle was injected in intravenous 5 min before the reperfusion. The ratio of infarct size to area-at-risk was significantly reduced in AM-001 treated animals (36±3%) when compared with that in vehicle treated mice (50±3%) (FIG. 15). Interestingly, although (R)-CE3F4 had a lower IC50 than AM-001 in the BRET sensor assay, and had been shown to efficiently and selectively inhibit Epac1-dependent biological action in vitro in various cellular models, this inhibitor (8 mg/kg body) failed to reduce infarct size, suggesting that CE3F4 might have a poor biodisponibility in vivo compared to AM-001 (FIG. 16). Altogether, these results demonstrate that acute injection of AM-001 is efficient to prevent myocardial reperfusion injury.

6. AM-001 Improved Cardiac Function During Chronic-AR Activation

The inventors next sought to determine whether AM-001 could provide improvement in cardiac function and remodelling during chronic p-AR activation. To this end, wild-type C57BL/6 mice were treated with either the non-selective R-AR agonist, isoproterenol (ISO) (60 mg/kg per day), or vehicle for 14 days in the presence of AM-001 (10 mg/kg i.p. from day 3 to day 14) or its vehicle. Liver and renal tissue samples from AM-001 treated mice did not display any apparent histological alterations as revealed with the hematoxylin and eosin (H & E) staining. In addition, plasma levels of alanine aminotransferase (ALT) and aspartate aminotransferase (AST) that are indicative of hepatotoxicity, were equivalent in each group (FIG. 17). Yet, fractional shortening (FS) as well as the ratio of left ventricular weight (LVW) to tibia length (LVW/TL) and heart rate were similar at baseline in vehicle and AM-001 treated mice (FIG. 18). Therefore, chronic injection of AM-001 had no toxic effects in animals and did not alter basal cardiac function under the experimental conditions used.

Chronic infusion of ISO resulted in a significant increase in LVW/TL ratios compared with control vehicle (FIG. 18). Conversely, injection of AM-001 3 days after the beginning of the adrenergic stress further decreased this parameter (FIG. 18). In addition, fibrosis was strongly decreased in AM-001-treated mice (1.1±0.5% vs 3.0±1.1% in Vehicle+ISO-treated mice) (FIG. 19). Importantly, after 14 days of ISO infusion the effect of AM-001 was associated with an improved cardiac function, as demonstrated by the higher FS in AM-001 treated mice than in vehicle treated mice (FIG. 20, top panel). Consistently, this better contractility observed in the presence of AM-001 during ISO treatment correlated with a decrease in left ventricular end-systolic internal diameter (LVIDs) and left ventricular end-diastolic internal diameter (LVIDd) (FIG. 20, lower panels). Finally, the inventors investigated whether AM-001 could modify the expression levels of G-protein receptor kinase 2 (GRK2) and GRK5. Indeed, both of these GRKs desensitize p-ARs and their upregulation in cardiac stress conditions participates in adverse remodeling and contractile dysfunction (24). As expected, ISO treatment increased GRK2 and GRK5 expression (FIG. 21). Interestingly, the upregulation of GRK5, but not GRK2 was significantly reduced in AM-001 treated animals.

Altogether these data show that AM-001 attenuates cardiac hypertrophy and fibrosis in response to chronic activation of 3-AR and improves cardiac function probably via the restoration of R-AR responsiveness.

In summary, these results concern the identification of a novel thieno[2,3-b]pyridine analogue as a specific pharmacological inhibitor of Epac1 that functions both in vitro and in vivo. They indicate that this compound may not only be a valuable pharmacological tool to explore physiological and pathological processes related to signalling pathways that are regulated by Epac1, but also has a great potential to be developed as a novel molecular therapeutic for relevant human diseases.

1. A method for treating and/or preventing a disease wherein the Epac protein is involved, wherein said disease is 5 a cardiac disease selected from the group consisting of: cardiac hypertrophy, cardiac arrhythmias, valvulopathies, diastolic dysfunction, chronic heart failure, ischemic heart failure, myocardial ischemia, reperfusion injury, myocarditis, hypertrophic and dilated cardiomyopathies, said method comprising administering to a patient in need thereof a pharmaceutical acceptable amount of a compound of formula (I), said compound having the following formula (I): 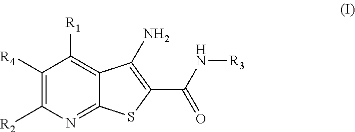
wherein:
R₁ is selected from the group consisting of:
H;
(C₂-C₂₀)alkyl;
(C₃-C₁₀)cycloalkyl;
3-10 membered heterocycloalkyl;
(C₆-C₁₀)aryl; and
5-10 membered heteroaryl;
wherein said alkyl, cycloalkyl, heterocycloalkyl, aryl and heteroaryl groups are optionally substituted;
R₂ is selected from the group consisting of:
H;
(C₁-C₂₀)alkyl;
(C₃-C₁₀)cycloalkyl;
3-10 membered heterocycloalkyl;
(C₆-C₁₀)aryl; and
5-10 membered heteroaryl;
or R₂ and R₄ together with the carbon atoms carrying them form a (C₃-C₁₀)cycloalkyl group;
wherein said alkyl, cycloalkyl, heterocycloalkyl, aryl and heteroaryl groups are optionally substituted;
R₃ is selected from the group consisting of:
H;
(C₃-C₁₀)cycloalkyl;
3-10 membered heterocycloalkyl;
(C₆-C₁₀)aryl; and
5-10 membered heteroaryl;
wherein said cycloalkyl, heterocycloalkyl, aryl and heteroaryl groups are optionally substituted; and
R₄ is selected from the group consisting of: H, —OH, —NRxRy and —C(O)ORz, Rx, Ry and Rz being independently of each other H or a (C₁-C₁₀)alkyl;
or R₂ and R₄ together with the carbon atoms carrying them form a (C₃-C₁₀)cycloalkyl group;
or its pharmaceutically acceptable salt, hydrate or hydrated salt or its polymorphic crystalline structure, racemate, diastereomer or enantiomer.

2. The method according to claim 1, wherein in formula (I) R₃ is selected from the group consisting of:
(C₃-C₁₀)cycloalkyl;
3-10 membered heterocycloalkyl;
(C₆-C₁₀)aryl; and
5-10 membered heteroaryl;
wherein said cycloalkyl, heterocycloalkyl, aryl and heteroaryl groups are optionally substituted.

3. The method according to claim 1, wherein in formula (I) R₁ is selected from the group consisting of:
H;
(C₆-C₁₀)aryl; and
5-10 membered heteroaryl;
wherein said aryl and heteroaryl groups are optionally substituted by one or more substituent(s) selected from the group consisting of —NR₇R₈, (C₁-C₁₀)alkyl and halogen atom; wherein R₇ and R₈ are independently of each other selected from (C₁-C₁₀)alkyl or H.

4. The method according to claim 1, wherein in formula (I) R₂ is selected from the group consisting of:
H;
(C₁-C₂₀)alkyl;
(C₆-C₁₀)aryl; and
5-10 membered heteroaryl;
or R₂ and R₄ together with the carbon atoms carrying them form a (C₃-C₁₀)cycloalkyl group;
wherein said alkyl, cycloalkyl, aryl and heteroaryl groups are optionally substituted by one or more substituent(s) selected from the group consisting of: (C₁-C₁₀)alkyl and halogen atom.

5. The method according to claim 1, wherein in formula (I) R₃ is a (C₆-C₁₀)aryl optionally substituted by one or more substituent(s) selected from the group consisting of: (C₁-C₁₀)alkyl and halogen atom.

6. The method according to claim 1, wherein R₄ is H or R₂ and R₄ together with the carbon atoms carrying them form a (C₅-C₆)cycloalkyl group.

7. The method according to claim 1, wherein in formula (I) R₁ is a phenyl group and/or R₂ is a thienyl group, said phenyl and thienyl groups being optionally substituted.

8. The method according to claim 1, wherein in formula (I):
R₁ is selected from the group consisting of:
(C₂-C₂₀)alkyl;
(C₃-C₁₀)cycloalkyl;
3-10 membered heterocycloalkyl;
(C₆-C₁₀)aryl; and
5-10 membered heteroaryl;
wherein said alkyl, cycloalkyl, heterocycloalkyl, aryl and heteroaryl groups are optionally substituted; and
R₂ is selected from the group consisting of:
H;
(C₁-C₂₀)alkyl;
(C₃-C₁₀)cycloalkyl;
(C₆-C₁₀)aryl; and
5-10 membered heteroaryl;
or R₂ and R₄ together with the carbon atoms carrying them form a (C₃-C₁₀)cycloalkyl group;
wherein said alkyl, cycloalkyl, aryl and heteroaryl groups are optionally substituted.

9. The method according to claim 1, comprising administering a pharmaceutical acceptable amount of a compound having the following formula (II): 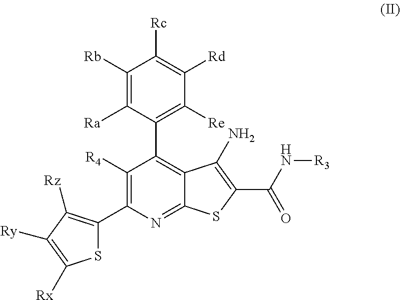
wherein Ra, Rb, Rc, Rd, Re, Rx, Ry and Rz are selected among the group consisting of: H, —OH, halogen atom, —C(O)OH, (C₁-C₁₀)alkyl, (C₁-C₁₀)alkoxy, and —NR₅R₆,
wherein R₅ and R₆ are independently of each other selected from (C₁-C₁₀)alkyl or H;
R₄ is selected from the group consisting of H, —OH, —NH₂ and —C(O)OH; and
R₃ is as defined in claim 1.

10. The method according to claim 1, wherein the compound has one of the following formulae:

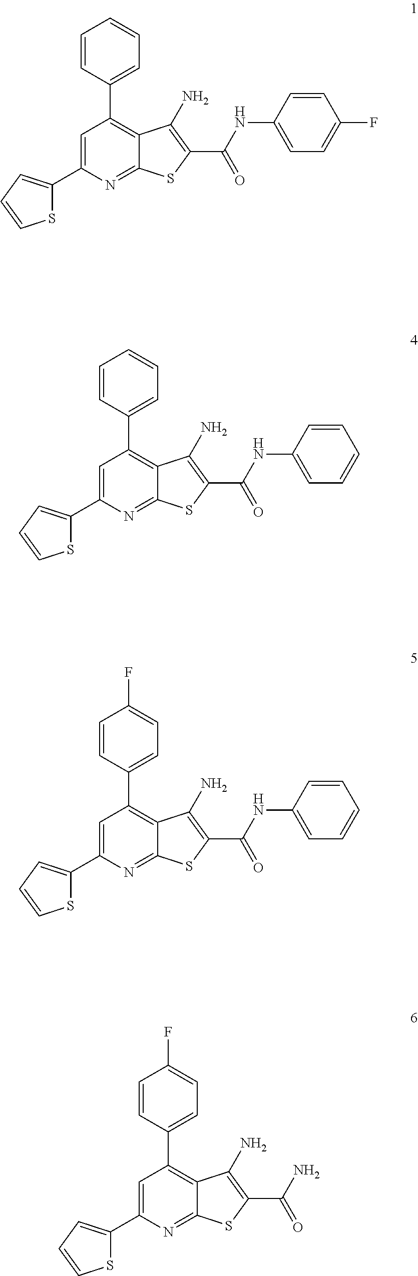 [SMILES: Nc1c(C(=O)Nc2ccc(F)cc2)sc2nc(-c3cccs3)cc(-c3ccccc3)c12 ; Nc1c(C(=O)Nc2ccccc2)sc2nc(-c3cccs3)cc(-c3ccccc3)c12 ; Nc1c(C(=O)Nc2ccccc2)sc2nc(-c3cccs3)cc(-c3ccc(F)cc3)c12 ; NC(=O)c1sc2nc(-c3cccs3)cc(-c3ccc(F)cc3)c2c1N]

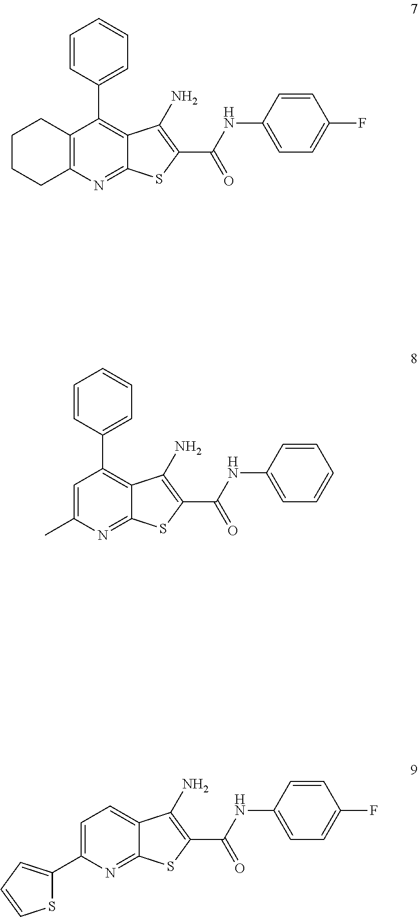 [SMILES: Nc1c(C(=O)Nc2ccc(F)cc2)sc2nc3c(c(-c4ccccc4)c12)CCCC3 ; Cc1cc(-c2ccccc2)c2c(N)c(C(=O)Nc3ccccc3)sc2n1 ; Nc1c(C(=O)Nc2ccc(F)cc2)sc2nc(-c3cccs3)ccc12]

11. The method according to claim 1, wherein the compound has the following formula:

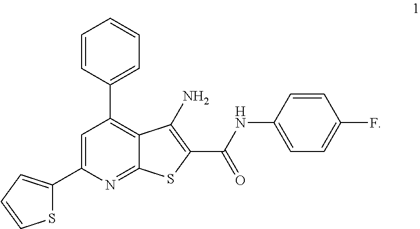 [SMILES: Nc1c(C(=O)Nc2ccc(F)cc2)sc2nc(-c3cccs3)cc(-c3ccccc3)c12]

12. A pharmaceutical composition comprising a compound having formula (I) as defined in claim 1, in association with at least one pharmaceutically acceptable excipient.

13. (canceled)
